SEQUENCE LISTING (17 sequences)

SEQ ID NO: 1 - DNA, 12 bp, Homo sapiens
atatatatatat

SEQ ID NO: 2 - DNA, 315 bp, Homo sapiens
aacctgagtctgccaaggactagcaggttgctaaccaccctgtgtctcagttttcctacctgtaaaatgaagatattaacagtaactgccttcatagatagaagatagatagattagatagatagatagatagatagatagatagatagatagatagatagataggaagtacttagaacagggtctgacacaggaaatgctgtccaagtgtgcaccaggagatagtatctgagaaggctcagtctggcaccatgtgggttgggtgggaacctggaggctggagaatgggctgaagatggccagtggtgtgtggaa

SEQ ID NO: 3 - DNA, 40 bp, Homo sapiens
tctatctatctatctatctgtctgtctgtctgtctgtctg

SEQ ID NO: 4 - DNA, 28 bp, Homo sapiens
tctatctatctatctatctgtctgtctg

SEQ ID NO: 5 - DNA, 44 bp, Homo sapiens
tctatctatctatctatctatctatctgtctgtctgtctgtctg

SEQ ID NO: 6 - DNA, 44 bp, Homo sapiens
tctatctatctatctatctatctgtctgtctgtctgtctgtctg

SEQ ID NO: 7 - DNA, 60 bp, Homo sapiens
tctatctatctatctatctatctatctatctatctatctatctgtctgtctgtctgtctg

SEQ ID NO: 8 - DNA, 64 bp, Homo sapiens
tctatctatctatctatctatctatctatctatctatctatctatctgtctgtctgtctgtctg

SEQ ID NO: 9 - DNA, 80 bp, Homo sapiens
tctatctatctatctatctatctatctatctatctatctgtctgtctgtctgtctgtctgtctgtctgtctgtctgtctg

SEQ ID NO: 10 - DNA, 67 bp, Homo sapiens
tctatctatctatatctatctatctatcatctatctatccatatctatctatctatctatctatcta

SEQ ID NO: 11 - DNA, 83 bp, Homo sapiens
tctatctatctatatctatctatctatcatctatctatccatatctatctatctatctatctatctatctatctatctatcta

SEQ ID NO: 12 - DNA, 75 bp, Homo sapiens
tctatctatctatatctatctatctatcatctatctatccatatctatctatctatctatctatctatctatcta

SEQ ID NO: 13 - DNA, 79 bp, Homo sapiens
tctatctatctatatctatctatctatcatctatctatccatatctatctatctatctatctatctatctatctatcta

SEQ ID NO: 14 - DNA, 87 bp, Homo sapiens
tctatctatctatatctatctatctatcatctatctatccatatctatctatctatctatctatctatctatctatctatctatcta

SEQ ID NO: 15 - DNA, 91 bp, Homo sapiens
tctatctatctatatctatctatctatcatctatctatccatatctatctatctatctatctatctatctatctatctatctatctatcta

SEQ ID NO: 16 - DNA, 95 bp, Homo sapiens
tctatctatctatatctatctatctatcatctatctatccatatctatctatctatctatctatctatctatctatctatctatctatctatcta

SEQ ID NO: 17 - DNA, 99 bp, Homo sapiens
tctatctatctatatctatctatctatcatctatctatccatatctatctatctatctatctatctatctatctatctatctatctatctatctatcta

(19) United States
(12) Patent Application Publication
(10) Pub. No.: US 2010/0114956 A1
(43) Pub. Date: May 6, 2010

(54) SYSTEM AND METHOD FOR INFERRING STR ALLELIC GENOTYPE FROM SNPS

(75) Inventors: Kevin McElfresh, Stafford, VA (US); Ronald G. Sosnowski, Coronado, CA (US)

Correspondence Address: DLA PIPER LLP (US), 4365 EXECUTIVE DRIVE, SUITE 1100, SAN DIEGO, CA, 92121-2133, US

(73) Assignee: Casework Genetics, Woodbridge, VA (US)

(21) Appl. No.: 12/578,420

(22) Filed: Oct. 13, 2009

Related U.S. Application Data
(60) Provisional application No. 61/195,988, filed on Oct. 14, 2008.

(51) Int. Cl. G06F 17/30; C12Q 1/68; C07H 21/00

(52) U.S. Cl. 707/780; 435/6; 536/23.1; 707/E17.014

(57) ABSTRACT

The present invention provides methods to infer STR allelic genotype from SNPs in a genome by obtaining statistical probabilities for the association of a plurality of SNPs in a genome with a Short Tandem Repeat (STR) locus allele for the genome to obtain a SNP constellation association value.

### CROSS REFERENCE TO RELATED APPLICATIONS

The present application claims priority under 35 U.S.C. §119(e) to U.S. Provisional Application Ser. No. 61/195,988, filed Oct. 14, 2008, which is herein incorporated by reference in its entirety.

### BACKGROUND OF THE INVENTION

1. Field of the Invention

The present invention relates generally to the genotype of an individual and more specifically to the use of SNP-STR associative patterns to determine an STR genotype of an individual in order to identify individuals from a biological sample.

2. Background Information

Sufficient genetic variability exists in plant, animal and microbial genomes to support using the genetic variants as a means of identifying the biological source of a sample. Human and other plant and animal genomes have been resolved to the point that individuals can be unequivocally identified by DNA analysis.

Short Tandem Repeats (STRs) in the human genome are currently used as the genetic variant marker for the absolute identification of an individual. They are difficult to analyze and their molecular makeup limits the technologies applicable to their analysis. Single Nucleotide Polymorphisms (SNPs) are simple variants technologically more amenable to determination. They are also better suited than STRs for mathematical analysis. However as a result of over 10 years of testing, massive databases exist for human identification based on STR markers. There are no such databases that use SNP variants as markers.

National and international databases have been established using STR alleles to uniquely identify biological samples. The Combined DNA Index System (CODIS) is used in the United States and Interpol and the Forensic Science Service (FSS) in the United Kingdom also have large STR databases. Much effort has been put into these databases and the number of profiles in them is over 5 million in the U.S. and greater than 10 million profiles in Europe. Therefore changing the databases from STRs to some other DNA marker is prohibitive even at costs of pennies per test. Further since many data points come from forensic samples that no longer exist, there is no possibility of comprehensively redoing the databases and retaining the maximum efficacy.

However, analysis of STR loci is technically difficult, making it slow, expensive, and requiring a sample quality that is greater than that sometimes obtained in a forensic or operational milieu. Conversely, there are numerous fast, cheap and easy commercially available methods for analyzing SNPs. This is because of the broad involvement of and deep interest in, SNPs over their roles in genetic disease and pharmacogenetics. This medical need has fueled a market pull and concomitant technology push to provide a surfeit of SNP detection methodologies. The methods for SNP detection are continually improving while conversely STRs are becoming less important as markers for genetic medicine and therefore less technological development effort is being applied to improve their detection. Many experts believe that given the size, the cost, and the intense labor requirements needed to validate new systems that the human STR identification databases will not change anytime in the near future. This means that human identification is at risk of being left behind technological advances in DNA analysis.

### SUMMARY OF THE INVENTION

The present invention is based on the discovery that one can infer STR allelic genotype from SNPs in a genome by obtaining statistical probabilities for the association of a plurality of SNPs in a genome with a Short Tandem Repeat (STR) locus allele for the genome to obtain a SNP constellation association value.

Thus, in one embodiment, a method and system are provided for inferring STR allelic genotype from SNPs in a genome including obtaining statistical probabilities for the association of a plurality of SNPs in a genome with a Short Tandem Repeat (STR) locus allele for the genome to obtain a SNP constellation association value. In another embodiment, this SNP constellation association value is compared with a database of STR locus alleles, wherein the output provides matches allowing identification of an individual from the sample.

In another embodiment, a system and method are provided for generating a SNP constellation for a genome including obtaining a plurality of SNPs in a genome that are associated with an STR type.

In an additional embodiment, a method and system are provided for inferring a genetic variant locus allele in a genome including obtaining statistical probabilities for the association of a plurality of SNPs in a genome with a genetic variant locus allele for the genome to obtain a SNP constellation association value. In one aspect, a database containing an SNP constellation of the invention is provided.

In a further embodiment, a computer system and method are provided including: a relational database having records containing a) information identifying the SNP constellation for a genome; b) information identifying a polymorphic locus allele; and c) a user interface allowing a user to selectively access the information contained in the records.

In an additional embodiment, a computer program product and method are provided including: a computer-usable medium having computer-readable program code embodied thereon relating to a relational database having records containing a) information identifying the SNP constellation for a genome; b) information identifying a polymorphic locus allele; wherein a SNP constellation association value is determined based on a) and b).

In a further embodiment, a computerized system and method are provided for inferring STR allelic genotype from SNPs in a genome including: receiving, by a computer, a plurality of SNPs of the genome; receiving, by the computer, a STR locus allele of the genome; and computing, by the computer, a SNP constellation association value associating the plurality of SNPs of the genome with the STR locus allele for the genome.

In an additional embodiment, a computerized system and method are provided for inferring a genetic variant locus allele in a genome, including receiving, by a computer, a plurality of SNPs of the genome; receiving, by the computer, a SNP constellation association value associating the plurality of SNPS of the genome with the genetic variant locus allele for the genome; and computing, by the computer, statistical probabilities for the association of a plurality of SNPs in the genome with genetic variant locus allele for the genome to obtain SNP constellation association value.

In a further embodiment, a computer system and method are provided for generating a SNP constellation for a genome, including: a server and a client connected by a network; an application connected to the server and/or the client by the network, the application configured for: obtaining a plurality of SNPs in a genome that are associated with an STR type computerized method for inferring STR allelic genotype from SNPs in a genome including: receiving, by a computer, a plurality of SNPs of the genome; receiving, by the computer, a STR locus allele of the genome; and computing, by the computer, a SNP constellation association value associating the plurality of SNPs of the genome with the STR locus allele for the genome.

### BRIEF DESCRIPTION OF THE FIGURES

FIG. 1 illustrates a system for inferring STR allelic genotype from SNPs in at least one genome, according to one embodiment.

FIG. 2 illustrates a comparison of STRs and SNPs in terms of the number of possible allele combinations and relative size of the target region.

FIG. 3 illustrates a method for inferring STR allelic genotype from SNPs in at least one genome, according to one embodiment.

FIG. 4 illustrates a polygenetic tree of the TPOX locus, one of the U.S. CODIS loci, drawn based on the frequency of the STR alleles in the Caucasian population.

FIG. 5 illustrates STR allele patterns that correspond to a SNP allele in the model system of FIG. 4.

FIG. 6 illustrates details related to how the SNP information can be compared to the STR locus allele information in order to obtain an associative value, indicating the probability that the organism is a match to the biological sample, according to one embodiment.

FIG. 7 illustrates an example of an STR locus allele used for human identification.

FIG. 8 illustrates an example of an STR locus and several of its alleles that contain internal microvariants.

### DESCRIPTION OF EMBODIMENTS OF THE INVENTION

The present invention provides methods for identifying Single Nucleotide Polymorphisms (SNPs) that are genetically associated with relevant STR loci in a manner that permits their use in inferring an STR-allelic makeup in a sample. These SNP STR-associative genetic patterns will be genomically equivalent to STR markers and can therefore be used to determine the STR genotype of an individual. Consequently SNP information may be used to infer the STR type which can then be used to search established STR databases to identify specific individuals or groups of people related to a biological sample.

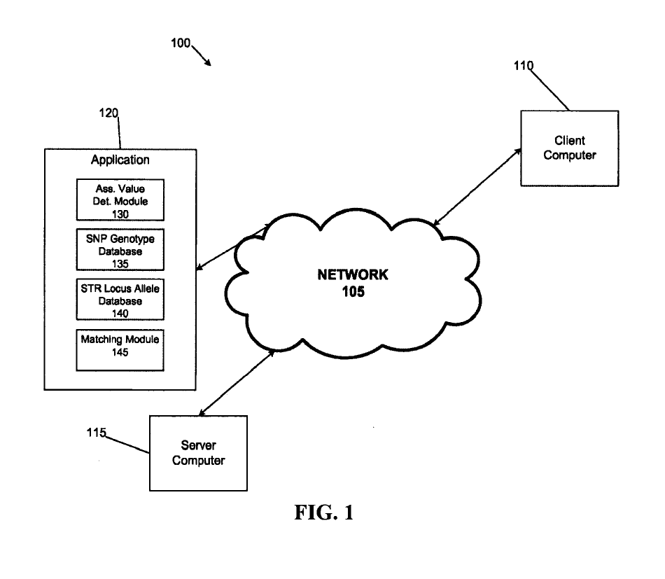

FIG. 1 illustrates a system for inferring STR allelic genotype from SNPs in at least one genome, according to one embodiment. The invention discloses an assay for the use of SNPs as a way of gaining knowledge of the STR alleles in a biological sample. Referring to FIG. 1, at least one client computer 110 can be connected to at least one server computer 115 over the network 105. At least one application 120 can be connected to the at least one client computer 110 and/or the at least one server computer 115 over the network. The at least one application 120 can comprise at least one associative value determination module 130; at least one match module 145, at least one SNP genotype database 135, and at least one STR locus allele database 140. It should be noted that the databases 135 and 140 can reside on application 120, or outside application 120. In addition, application 120 can reside on the client computer 110 and/or the server computer 115. Furthermore, many additional databases and modules can be utilized by application 120, and can reside on application 120 or outside application 120.

The at least one associative determination module 130 can determine a statistical probability of SNP-STR co-inheritance designated as an associative value. A first component of the associative value is the linkage disequilibrium between the STR variable repeat region and nearby SNPs. (This is described in more detail below.) Another component of the associative value is the differential mutation rates between STRs and SNPs. (This is also described in more detail below.) The associative value may be determined empirically by scanning databases or by direct experimentation. Additionally the associative value may be determined mathematically from data gained in the empirical analysis.

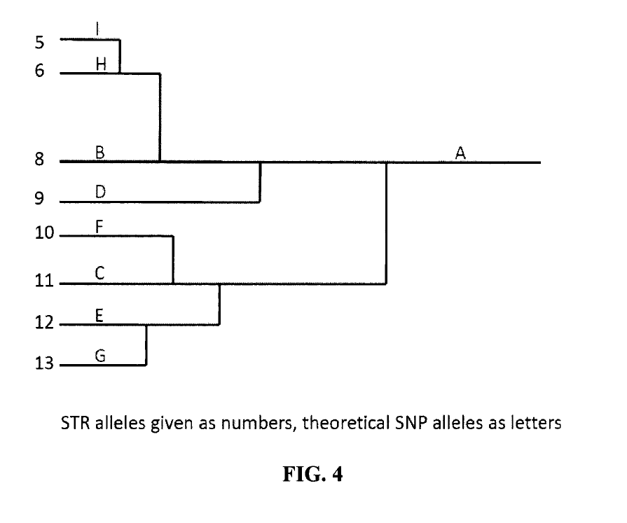

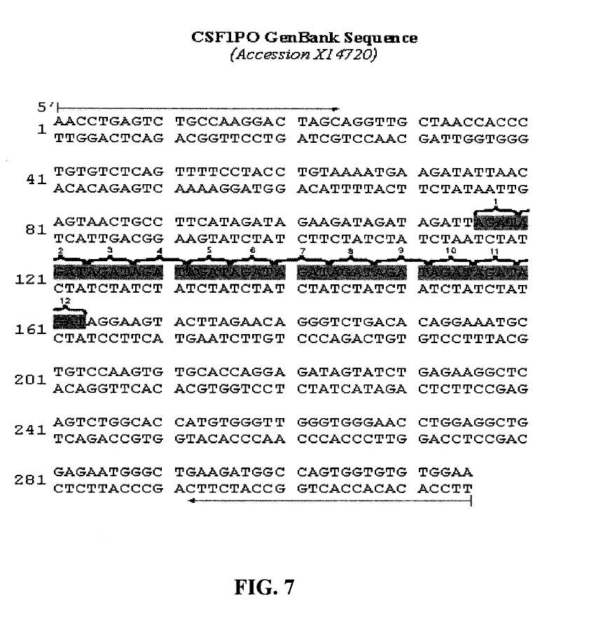

To help explain how the associative value is determined, FIG. 4 illustrates a phylogenetic tree of the TPOX locus, one of the U.S. CODIS loci is drawn based on the frequency of the STR alleles in the Caucasian population. The numbers 5 through 13 are the representation of the STR alleles while the letters are representation of the SNPs. The invention consists of sets of SNPs that are associated with STR loci that can be used to determine associative STRs to sets of SNP patterns thus providing a genetic bridge between SNP variants and STR variants The bridge will be both genetic and statistical. Consequently, from a composite SNP type, the STR type within a database can be inferred and thus the STR type can be use for a database search thus preserving the STR databases utility while taking advantage of the newer technological capabilities of SNP technology FIG. 7 illustrates an example of an STR allele, locus CSF1PO, allele 12.

For the invention to be enabled, a one to one association of SNP pattern with an STR allele is not strictly necessary. For example: a SNP STR-associated genetic pattern might be associated with THO1 6, 7 and 8 but not 9, 10 or 10.1. Doing the same type of association for all 13 CODIS loci, and perhaps some others, one would search the CODIS database for entries that have for example:

ThO—6, 7, 8

VWF—5, 6, 7

D21—11, 12

This would result in selection of a group of individuals who could have contributed the biological sample. Other forensic data (location of crime versus location of individual) could be used to further narrow the number of individuals who might match. From this pool of possible matches, individuals could be analyzed for SNP patterns used in determining the STR-SNP associative values to confirm their connection to the biological sample. This triage of genetic relevance results in an effective means of searching STR databases using only SNP data.

In one embodiment, the SNP association value is combined with genetic phenotype information. For example, a genetic pigmentation trait of a subject can be determined. For example, a nucleic acid sample or a polypeptide sample of a subject is utilized to identify a single nucleotide polymorphisms (SNPs) that, in combination with the SNP association value, allow an inference that includes a genetic pigmentation trait such as hair shade, hair color, eye shade, or eye color, skin shade/color and further allows an inference to be drawn as to race. As such, the compositions and methods of the invention are useful, for example, as forensic tools for obtaining information relating to physical characteristics of a potential crime victim or a perpetrator of a crime from a nucleic acid sample present at a crime scene, and as tools to assist in breeding domesticated animals, livestock, and the like to contain a pigmentation trait as desired. Further, genetic phenotypes that can be used in combination with an SNP association value of the invention include genetic diseases (e.g., risk of age-related macular degeneration; Huntington's disease; sickle cell anemia) (see for example US 2006/0263807; US2008/0193922). It is further contemplated that in order to protect personal genetic information, these data would be tightly controlled and released to officers of the court, for example.

An analogy to the present invention may be seen in the consideration of electrical conductivity. Materials are commonly referred to as either conductors or insulators. Copper, for example, is normally considered a conductor, and cloth is normally considered an insulator. Cloth can be found as an insulator in old wiring. However, if cloth is compared with numerous plastic materials or glass, it has a greater capacity to conduct electrons than those materials. Therefore cloth is a conductor relative to glass. Consequently, conductance is a differential movement of electrons along a path. However, in the case of a short circuit, the path of electrons is disrupted and the voltage will be lost or reduced to the point that the differential conductors are functionally equivalent. The parallel to that in this invention is that geneticists commonly consider that SNPs display a functionally null mutation rate in comparison to STRs and that therefore mutations for the two types of genetic variants will arrive at the destination of the modern genotype at different rates. However, since mutations especially in medically or physiologically relevant areas of the genome cause a drop or complete loss of genetic fitness of the organism—in effect a genomic short circuit—the net effect is a lack of apparent genetic linkage even within a centimorgan of genomic distance. Therefore dogma dicates there is no practical utility in using SNPs for genetic identification. This invention teaches that this commonly held belief is incorrect and that there is a genetic association between STRs and SNPs that is determinable and useful in the context of DNA identification.

It has been argued that, in order for an organism to remain fit in a genetic sense with regard to high mutation rates, there is a truncated selection mechanism to balance the mutation rate: in effect, removing mutations via genetic death (PNAS 1997 94:16 pp. 8380-8386). This is important in regions of the genome that are phenotypically relevant. In the case of phenotypic relevance, allelic associations may be lost as a function of the truncated selection mechanism. The present invention discloses the reverse—that since there is no fitness related constraint on the genetic regions used for STR human identification, the SNPs and STRs have filtered through the population from the time that neo-modern human genomes effectively fixed on 23 chromosome pairs. This time frame is long enough to have developed associations as a result of population dispersement. Further, the present invention, when viewed as an evolutionary snapshot of only a few to several generations is generally insulated from additional ongoing mutations.

The feasibility of this approach may be evidenced by the novel consideration of two current means of determining an individual's lineage. Autosomal SNPs are used in determining an individual's human population origin (23 and me, DNAPrint Genomics). Therefore SNPs can be associated with an evolutionary path resulting in a group of people with a genetic “likeness”. STRs have also been used to predict an individual's ethnicity (The DNA Ancestry Project, The Genographic Project). This indicates that STR alleles are associated with a selected population as well. In fact SNPs and STR alleles may be associated with the same selected population. It follows then that the SNPs and STR alleles that are associated with the same population must be associated with each other. This unique “A=B, B=C, A=C” perspective is a corollary consistent with the theses behind the invention. This invention determines the association between SNPs and STR alleles to derive specific associative values and use them for human identification applications.

A SNP STR-associative genetic pattern may comprise as few as a single SNP or as many as can be associated with an STR locus in a non-random fashion. (FIG. 4 presents a theoretical simplistic case.) From FIG. 4, an STR allele 5 would be associated with an SNP constellation of AI.

A SNP STR-associative genetic pattern may include any genetic variant marker for which an associative value can be determined. These may include but are not limited to: SNPs in regions flanking target STR hypervariable region, SNPs that are biallelic, SNPs that are triallelic, SNPs that are tetrallelic, insertions, deletions, simple repeat variants, SNPs within target loci repeat units of the target STR hypervariable region, non-target STRs, copy number variations, translocations, methylation modifications, deacetylation modifications, epigenetic markers and any other determinable genetic variants. In one aspect, the genetic variant allele locus is amelogenin. In another aspect the locus is associated with a disease or disorder.

In an alternative embodiment, while association values for SNPs in combination with STRs are exemplified herein, other polymorphisms or genetic variation can be used with STRs including but not limited to INDELS, copy number variations (CNVs), hypervariable regions and the like.

An embodiment of this invention is the exclusion of STR determination in the identification of individuals in an STR database who may be associated with a biological sample (e.g., blood, semen, vaginal swabs, tissue, hair, saliva, urine, bone, skin and mixtures of body fluids or tissue). This invention therefore makes SNP technology “back-compatible” with the vast STR databases.

The invention has applications for use with STRs not included in CODIS and is equally compatible with other non-CODIS databases such as the databases used by Interpol, FSS and others.

The invention also has applications for use with STRs that are unrelated to forensics or human identification such as Genome Wide Association Studies.

The invention also has applications for use with repeat loci that are made up of repeat units varying by the number of nucleotides, including but not limited to: di-, tri-, tetra-, penta-, hexa-, hepta-nucleotide repeats, and repeat units having greater numbers of nucleotides.

The also invention has applications for use with repeat units that have varying conformations, including but not limited to: head to tail, head to head, tail to tail and all combinations of the preceding repeat unit arrangements.

The invention also has applications for use with non-human individual identification. Non-human identification may include animals (domestic or wild), plants, insects, invertebrates and microbes.

As mentioned above, one component of the associative value is the linkage disequilibrium between the STR variable repeat region and nearby SNPs. Another component of the associative value is the differential mutation rates between STRs and SNPs. These two concepts are described in more detail below.

A centimorgan is a measure of genetic “distance” corresponding to a 1% recombination rate. In humans it is about 1 million bases. SNP frequency is about 1 in 1000 bases so there would be 1000 SNPs for every 1% of recombination. This means that genetic variants that are contained within that sequence space have a 99% probability of being passed on to progeny as an intact unit. While the invention is not limited to any number of bases it could include, for example, the analysis of 1000 bases on each side of the STR locus for each allele. See, for example, FIG. 4.

The mutation rates for STRs are 2 for every 10 reproductive events, while SNPs change at a rate of 2 in 103 to 104 events. It is an advantage for this invention that the SNP rate is so low since this means the SNP haplotype will not vary much. Yet even the STR mutation rate is low enough to permit ethnic association with STR genotypes. Underhill and colleagues (2003) use this disparity in mutation rates to do phylogenetic analysis of genetic variants. This comprehensive analysis of all human genetic evolution surveys 1000's of generations of the human population over millions of years. On that time scale, the differential mutation rate is significant. However for human identification analysis it is only necessary to assess 1, 2 or at most 3 or 4 generations, essentially the current human genome carried by live individuals. In this evolutionary snapshot analysis, mutation rates are much less impactful as causes of additional variation.

The associative values will be affected by both linkage disequilibrium and mutation rates. This invention may use empirical data derived from existing databases such as the HAPMAP project to determine the associative values. Experiments, such as sequence determination of select populations may be carried out with the specific intent of elucidating associative values. Alternatively mathematic functions or algorithms may be used to determine associative values either independently or in combination with empirically derived associative values.

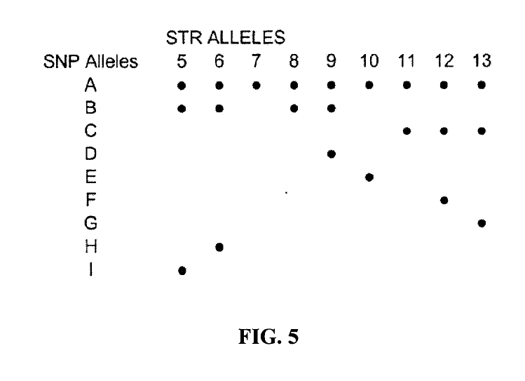

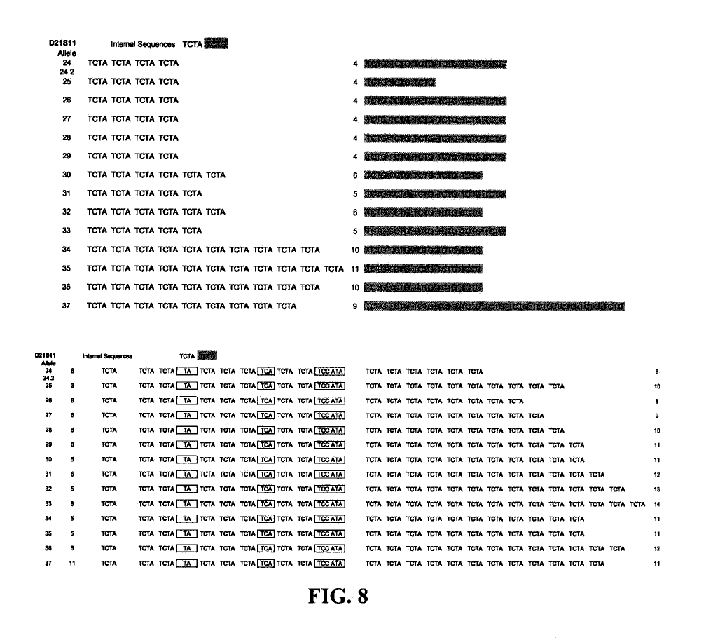

A SNP pattern may be associated with more than one STR allele (see, e.g., FIGS. 5 and 7) or more than one SNP pattern may be associated with a single STR allele (see, e.g., FIG. 8). We teach here that this association value may be determined for each case empirically and assigned to each SNP-STR association. By combining SNP triaged STR loci, as for example, in the Combined DNA Index System 13 STR loci, we will be able to match individuals in databases with STR genotypes solely on the basis of SNP sequence information.

Further, we may also use frame of reference SNPs that are associated with genotypes that are not associated with STRs. Therefore we will be able to say that in the genotypic background identified by SNP pattern X, SNP pattern Y is linked with STR allele TPOX 6. However in the genotypic background identified by SNP pattern Z, the same SNP pattern Y is associated with STR allele TPOX 10. The non-STR-SNP genotypic background may due to ethnicity, disease resistance or other genetic features that are stable on an appropriate time scale. These frame of reference SNP patterns can come from autosomal, Y, and/or mitochondrial genetic sources and may be the same as those used for lineage testing (23 and me, DNAPrint Genomics). Alternatively, non-STR-SNPs may be found that sort with STR-SNPs, independent of the factors cited above.

In one embodiment, 1,000,000 bases containing 1000 SNPs on either side of the STR, may be analyzed resulting in 2000 SNPs available to provide association for each STR locus. For 13 STR loci there will then be ˜26,000 SNPs. The plurality of SNPs can be from about 10 to 30,000,000; 30,000 to 3,000,000; 300,000 to 3,000,000; or 3,000,000 to 30,000,000 or any combination thereof. Technologies capable of analyzing that many SNPs have been available since 2002 (e.g., Affymetrix and 454). Today such technologies are becoming commonplace. Several products (e.g., 454, ABI, Affymetrix and Illumina) have the capacity to rapidly and inexpensively type 2,000,000 bases. Newer technologies, such as Pacific Biosciences, Opgen and other single molecule sequencing technologies, are rapidly coming to market. While earlier technologies were capable of enabling the invention as early as 2002, these newer technologies promise ever more efficient means of handling the throughput required for this invention.

Whole genome sequencing technology is rapidly progressing. These technologies are well suited to this invention. It is further contemplated that, while only a subset of a genome is necessary to determine STR-associative genetic patterns, it may be more practical to attain whole genome sequence information. The practicality may come from the development of systems and kits that are highly refined for whole genome sequencing, while being cumbersome for attaining a subset of the genome. It is recognized that whole genome sequencing may be a practical technology for this assay.

Mixtures are a very difficult issue for human identification studies. They are common in criminal investigations as evidence is distributed through unregulated actions. As SNP analysis is rapidly progressing, mathematical methods are being developed that aid resolution of mixtures. Such mathematical methods that resolve mixtures may also be used to determine associative factors for relating SNPs with STRs.

Additionally, the demand for sequence variation analysis at the single nucleotide level has led to computative products that are specific for SNPs but not STRs. These will work in combination with the instant invention.

In one embodiment, a single SNP pattern will be associated with a single STR allele. In another embodiment, the association between the SNP and STR locus may be that more than one SNP pattern is associated with a single STR allele. In a further embodiment, the association between the SNP and STR locus may be that a single SNP pattern is linked with more than one STR allele.

The present invention is an assay to determine the genetic association between genetic variants. In a preferred embodiment this assay comprises information associating SNP patterns with STR alleles.

An association factor that can be determined for each SNP-STR combination is contemplated. This weighted value will be used to search the CODIS and other databases making SNP STR-associated genetic pattern typing back-compatible with STR databases. The predicted outcome of such a search, in one possible scenario, is that more than one individual who is a possible match for SNP analysis of a biological sample. may be identified. In this case other relevant information such as proximity of the individual to the event, physical description, cultural characteristics and other factors known to criminal investigators may be used to narrow the number of possible suspects. Ultimate identification of the individual associated with the biological sample will be by typing all persons in the final suspect pool for the STR-associative genetic pattern found in the sample.

In one aspect of the invention, a SNP association value can be used in combination with non-genetic information to identify individuals. For example, in the context of forensic studies in a criminal investigation, information such as whether an individual is incarcerated, whether they have a certain shoe size or certain weight range, whether the suspect is a man or woman, and the like can be utilized to further assist with identification of an individual.

Differential SNP/STR mutation rates perform cross correlation using signal processing algorithms, and Population Frequencies. There are three factors that are combined in a novel way in this invention. First, the unequal mutation rates of SNPs and STRs are considered fundamental to the analysis of the correlation of the STR type to the SNP type. Second, signal processing algorithms are the methods used to analyze the SNP data. Third, population frequencies of the SNPs are the additional information that allows the likelihood of the STR type to be completed.

With respect to differential mutation rates, the molecular mechanisms that drive mutation differ between SNPs and STRs (see, e.g., Mahtani, M. M. and Willard, H. F. (1993)). A polymorphic X-linked tetranucleotide repeat locus can display a high rate of new mutation, which has implications for mechanisms of mutation at short tandem repeat loci (see, e.g., Hum. Mol. Genet. 2: 431-437). Mountain et. al. (2002) pointed out that differential mutation rates were capable of examining the evolutionary history of a SNP/STR system using a single SNP linked to single STR. Further, they did not infer the STR type using the SNP as is done in this invention. This invention looks at multiple SNPs genetically linked to an STR such that the pattern and frequency of the SNPs associated with the STR locus will allow the inference of the unknown STR type. This is necessary as the technology for SNP analysis is significantly more sensitive than the technology for analysis of STRs when considering the typical crime scene sample which can contain highly degraded DNA. The likelihood of degradation impacting 13 STR loci is far greater than the degradation impact on a million (for example) SNP loci. When analyzing SNP loci, even a loss of 50% will leave more than enough intact or non-degraded SNP loci to allow for an unambiguous identification. Loss of 50% of STR loci from a sample would impact whether there was enough information to allow identification. Thus in a forensic sample, it is likely that the classical STR analysis alone would not yield results, while a SNP analysis would in fact provide sufficient information. (However, only the STR type would be searchable in a felon database).

FIG. 8 provides an illustration of this using current genotype information from an allele, D21S11, containing many microvariants. The left column indicates the allele designation, reflecting the number of tetrameric repeats present in various alleles. The non-whole number values indicate alleles where less than a complete tetrameric unit (e.g., only five, three or two bases) exists. These partial repeat units are generally insertions or deletions of bases, and may be generated by the same mechanisms as SNPs are generated. In databases of the current living and recently deceased human population, these microvariants are conserved. The present invention teaches the use of SNPs associated with the STR alleles and these data exemplified in FIG. 8 confirm that mutations other than addition or removal of intact tetramer repeat units can reliably associate with an STR allele. Therefore it follows that SNPs associated with specific STR alleles will also be conserved in the time frame that is relevant to forensic human identification.

The differential rate in mutation between STRs and SNPs means that there are going to be different associations of SNPs and STRs in different ethnic backgrounds, and in different STR allelic groups. For example, allele 7 of the CODIS STR TPOX, has not been seen in the Caucasian population, but exists in the differential frequencies of 0.7% in the Hispanic population, and 2% in the African populations. Within coding regions in a genome there is evolutionary constraint on mutation since almost all mutations in these areas are deleterious to the fitness of the organism, which in this case is a human. However, the forensic CODIS loci are chosen to be free of apparent phenotypic impact and therefore are also free from the selection pressure against mutations being maintained in the population. This means that. over the course of human evolutionary history, the STR and SNP mutations have been accumulating at different rates and are therefore going to group themselves into unique combinations.

From a practical application view with regard to this invention, it means that there will be an array of SNPs associated with the STR genetic sequence (both within and without) that will be available for correlation to groups of alleles and to individual alleles. The mathematical implications of this aspect of the invention are outlined below.

The following Examples are illustrative embodiments of the invention and are not intended to indicate any limitations relating to methods of determining genetic variation, instrumentation, technology, types of genetic markers, data analysis, data interpretation, statistical analysis or any other aspect of generating STR-associative genetic patterns and the like.

### Example 1

### Cross Correlation Using Signal Processing Algorithms

The overall process of collecting a DNA sample and processing it produces a two dimensional electropherogram of rfu (relative fluorescent units) values or in the case of SNPs, spot intensities that are interpreted as allele calls. For the purposes of this invention, we will use the conventional forensic rfu terminology to mean either STR electropherogram peak intensity or SNP array spot intensity, Current forensic DNA analytic techniques use only one of these dimensions, allele call, while generally ignoring the rfu values. Limiting one's attention to the allele calls while ignoring rfu or intensity values negates the contribution of multiple identical alleles, i.e. dosage, but is in keeping with the validated interpretation guidelines of standard forensic practice. In order to utilize the other dimension, rfu values, it is necessary to have a model describing the relationship between the input, the amplified DNA, and the output, the electropherogram. Again for the purposes of this invention, the term electropherogram will be used to mean the trace from an STR test or the array of intensities from an SNP test. Each from the standpoint of this model is equivalent. That process consists of several separate and distinct steps. One way to model such a process is to analyze each step in the process, formulate a description of that step, and then cascade the processes. An alternative approach that has proven successful in a wide range of physical and chemical processes, from communication in the presence of noise to the interpretation of photographs from space, is the application of stationary linear system analytic techniques. In this approach all of the individual steps are lumped together forming the “process” or a “black box”. Signals are placed in the “black box” and results come out of it. In our case the signal is an electropherogram. System analysis is limited to determining the relationship between input and output ignoring the details of the internal processes.

That entire process is successfully treated by the mathematical modeling proposed

$$\begin{matrix}Input & Yields & Output \\ δ ( x ) & \to & s ( x )\end{matrix}$$

Here, s(x) represents the spread function, and x is the molecular weight, measured in base pairs (bp) of the STR system or the array location of the SNP. Using these concepts we may define a stationary linear system. Throughout this discussion, we will use the term delta function, δ(x), to indicate a function which has the value zero everywhere except at x where it has the value 1. Mathematically, we ping the black box with a very sharp input, a delta function, and observe the resultant output, “ringing”. For each DNA sample input, there will be a set of output electropherograms, nτ(x); here n varies from 1 to n max and indicates which dye was used for the STR electropherogram (since multiple STR alleles can have the same molecular weight but are different due to the dye) or the array location of the SNP. The function is of the form:

nτ(x)=nΣiaᵢsn(x−xᵢ) n=1 to κ (1)

Here nΣi indicates the summation over the subscript i for the nth dye/SNP electropherogram; sn(x) denotes the spread function of the system for the nth dye electropherogram; xᵢ is the location of the ith peak in the respective electropherogram (or the SNP array location) and aᵢ is the amplitude of the ith peak. κ is the number of the SNP system or the dye of the STR system. This format is required since in general the spread functions in each dye electropherogram may be different from the others and in the case of mixed DNA samples the amplitudes of the peaks will vary. For the sake of simplifying this discussion we will work exclusively with a single electropherogram, reserving the expansion to include all dyes/SNPs. That the calculations must be repeated mutatis mutandis for each dye is implied. For single DNA samples aᵢ=aⱼ for all i and j; that is the amplitudes of all of the peaks in a single dye electropherogram are equal. There is an exception when a “doublet” occurs but for the case of a single DNA sample there is no loss of generality in including the secondary peak in the set. The peaks, maxima of the individual spread functions, are located at the points xᵢ determined by the equation s′(x−xᵢ)=0. Since they are all equal, the amplitudes may be normalized to 1. From nτ(x) we construct the identifier, nΩ(x), given by

nΩ(x)=nΣiδ(x−xᵢ). (2)

Since there are no zero elements in the DNA sample, we may define:

nΩ(0)=nN

where nN is the number of peaks in the nth dye electropherogram or the array locations of the SNP. Each SNP array therefore will have a unique identifier as will each STR electropherogram. Consequently there will be one SNP and one STR identifier that exactly correlate with each other and a single individual and therefore the STR type can be determined by the identifier generated for the SNP. This is the associative value. The exact correlation of the two identifiers will be determined empirically.

Searching a data base to find a match to a suspect DNA sample is analogous to searching through a series of messages, μn(x), to determine if a particular signal, f(x), is embedded in one or more of them and if so where it is located. The simplest such search is to cross correlate f(x) with each μn(x). If there is a match for f(x) in μ(x), the correlation will peak at its position. Mathematically, that operation is represented by the equation:

C(x)=∫f(σ)μ(σ−x)dσ. (3)

In the case of DNA analysis the signals must have not only the same shape, but also the same origin. Furthermore, both f(σ) and μ(σ) are discrete functions. Under these circumstances we will see that the cross correlation integral reduces to discrete products and summations.

### Example 2

### Population Frequencies of the SNP Alleles with Regard to the STR Alleles

It is clear that the SNP mutations will span the entire evolutionary history of the STR mutations. That is, there will be SNPs that are ancient and therefore found in all STR alleles and newer SNP mutations that are in a subset of the STR allele groups. This is important in the differentiation of the SNPs that overlap allele groups and can be dealt with simply using the Hardy-Weinberg (HW) population probabilities. For example, in a SNP result that clearly defines TPOX allele 11 but overlaps the TPOX alleles 6 and 8, the question is which TPOX allele is it? 6 or 8? The answer is based on the population frequency of the possible combinations. The HW probability is calculated as 1/2(pi*pj) where i≠j. In the Caucasian population the 11,6 combination has a probability of 1 in 1041 (using published STR allele frequencies) while the 11,8 combination has a probability of 1 in 4. Since the 11,8 combination has the highest probability of existence, the first result will be listed as 11,8. Given that these are probabilities, it is essential to note that the rare combination will be possible, and if based on the other SNP results the rare combination is indicated, then the strength of the identification will be that much stronger if not in fact definitive.

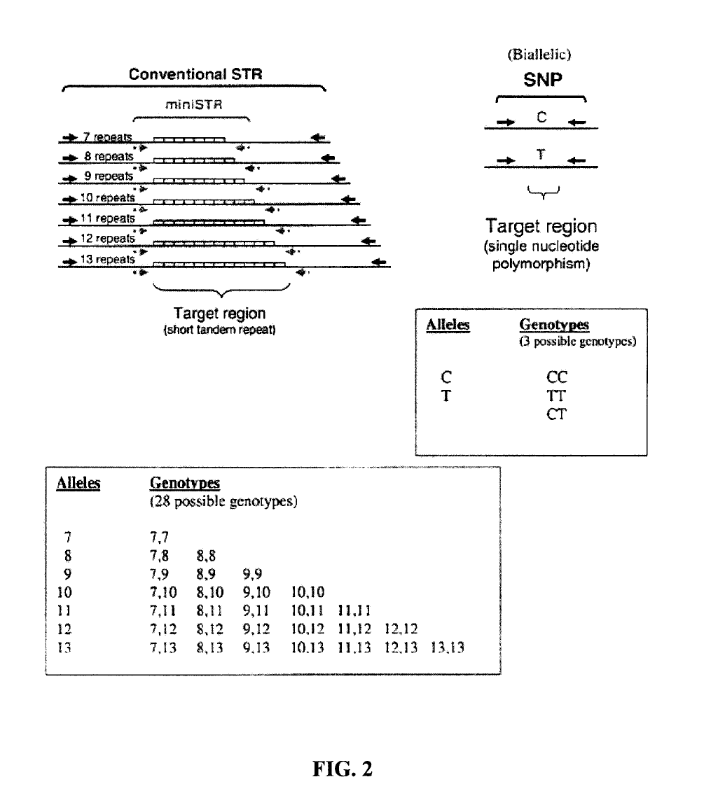

FIG. 2 illustrates a comparison of STRs and SNPs in terms of the number of possible allele combinations and relative size of the target region. Short Tandem Repeats (STRs) (used, e.g., in forensic DNA tests) are any short, repeating DNA sequence. For example, the DNA sequence ATATATATATAT is a STR that has a repeating motif consisting of two bases, A and T. DNA has a variety of STRs scattered among DNA sequences that encode cellular functions. Organisms vary from one another in the number of repeats they have, at least for some STR loci. For example, person #1 may have type 1 “ATATAT” at a particular locus while person #2 may have type 2 “ATATATATATAT” at the same locus. Single nucleotide polymorphisms (SNPs) are DNA sequence variations that occur when a single nucleotide (e.g., A, T, C, or G) in a genome sequence is altered. For example, a SNP might change the DNA sequence AAGGCTAA to ATGGCTAA. For a variation to be considered a SNP, it must occur in at least 1% of the population.

SNPs can be used to determine an individual's human population origin (see, e.g., 23 and me, DNAPrint Genomics). SNPs can be associated with an evolutionary path resulting in a group of people with a genetic “likeness”. STRs can also predict an individual's origin (See, e.g., DNA Ancestry Project, the Genographic Project). STR alleles can be associated with selected populations. In fact, SNPs and STR alleles may be associated with the same selected population. Thus, the SNPs and STR alleles that are associated with the same population must be associated with each other (A=B, B=C, thus A=C). In one embodiment, the association between SNPs and STR alleles can be discovered. This can be beneficial because SNP information is often easier to obtain, but significant STR databases exist.

Abundant SNP loci have been characterized and studied in various human populations. In addition, only a single nucleotide needs to be measured with SNP markers, whereas an array of nucleotides (sometimes hundreds of nucleotides in length) needs to be measured with STR markers. SNPs also have mutation rates 100,000 times lower than STRs. Thus, SNPs are more stable.

Analysis of STR loci can be more difficult, slow, and expensive than that required for analysis of an SNP. In addition, analysis of STR loci can require a sample quality greater than that required for analysis of an SNP. This can be because SNPs have had more research due to their roles in genetic disease and pharmacogenetics, which has resulted in multiple SNP detection methodologies.

As a result of years of testing, massive databases exist for human identification based on STR markers to uniquely identify biological samples. There are no such databases using SNP variants as markers. Changing the database from STRs to some other DNA marker (such as SNP) is prohibitive. Further, since many data points come from forensic samples that no longer exist, there is no possibility of comprehensively redoing the databases and retaining the maximum efficacy.

Thus, associating SNP information with STR information can be very beneficial. For example, population frequencies of the SNP alleles can be compared with the STR alleles. Because SNP mutations happen less often than STR mutations, the SNP mutations will span the entire evolutionary history of the STR mutations. That is, there will be SNPs that are ancient and therefore found in all STR alleles and newer SNP mutations that are in a subset of the STR allele groups. This can be important in the differentiation of the SNPs that overlap allele groups and can be dealt with using, for example, Hardy-Weinberg (HW) population probabilities.

For example, in an SNP result that clearly defines TPOX allele 11, but overlaps the TPOX allele 6 and 8, does 6 or 8 apply? The answer can be based on the population frequency of the population combinations. The HW probability can be calculated as 1/2(pᵢ*pⱼ) where i≠j. In the Caucasian population, the 11,6 combination has a probability of 1 in 1041 (using published STR allele frequencies), while the 11,8 combination has a probability of 1 in 4. Because the 11,8 combination has the highest probability of existence, the first result can be listed as 11,8. Given that these are probabilities, it is essential to note that the rare combination will be possible, and if based on the other SNP results, the rare combination is indicated, then the strength of the identification will be that much stronger if not definitive.

It should be noted that the STR locus allele can comprise at least one Combined DNA Index System (CODIS) database STR loci; or any other type of STR loci (e.g., non-CODIS database (e.g., Interpol, FSS) STR loci); or any combination thereof. For example, in one embodiment, the STR loci can be selected from the following group: TH01, TPOX, CSF1PO, vWA, FGA, D3S1358, D5S818, D7S820, D13S317, D16S539, D8S1179, D18S51, and D21S11. In another embodiment, the STR loci can be selected from the following group: TH01, TPOX, CSF1PO, vWA, FGA, D3S1358, D5S818, D7S820, D13S317, D16S539, D8S1179, D18S51, and D21S11.

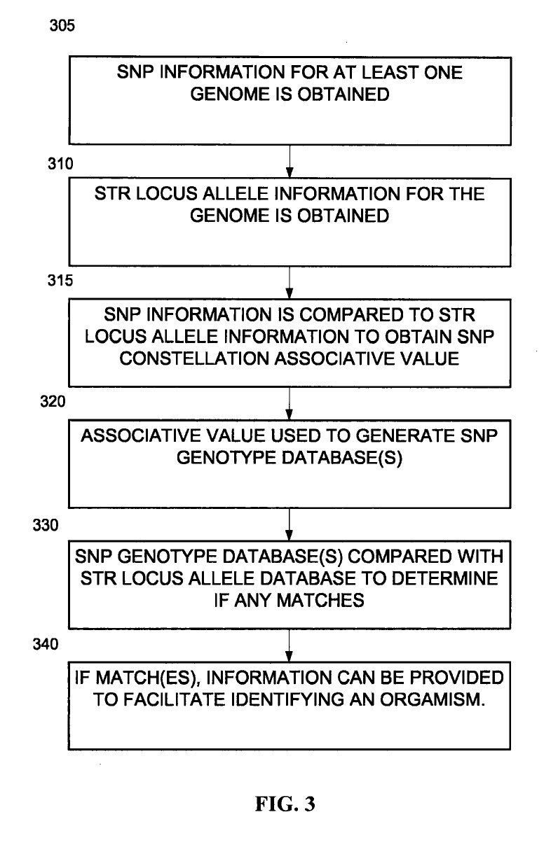

FIG. 3 illustrates a method for inferring STR allelic genotype from SNPs in at least one genome, according to one embodiment. In 305, SNP information of at least one genome can be obtained. In 310, Short Tandem Repeat (STR) locus allele information for the genome can be obtained, from, for example, a sample from an organism. The STR locus allele information can be used as genetic variant markers for the identification of an individual. Note that the sample (e.g., biological sample, nucleic acid-containing sample) can comprise: fingerprint, blood, semen, vaginal swabs, human tissue (e.g., single type, mixture), hair, saliva, urine, bone, skin, or body fluid (e.g., single type, mixture), or any combination thereof. In addition, the sample can be from more than one organism. For example, the sample can be blood from several people from a crime scene.

In 315, the SNP information can be compared to the STR locus allele information in order to obtain at least one SNP constellation associative value (also referred to a “statistical probability of SNP-STR co-inheritance” or “genetic variant locus allele information”). In one embodiment, the associative value can be determined by different mutation rates, linkage disequilibrium, insertion, deletion, repeat variant, copy number variant, translocation, methylation modification, deacetylation modification, or epigenetic marker, or any combination thereof. The associative value can be determined by scanning databases (e.g., the HAPMAP project); by direct experimentation (e.g., sequence determination of select populations); or by mathematic formulas; or by any combination thereof.

Referring to FIG. 4, a Phylogenetic tree of the TPOX locus, one of the US CODIS loci, is illustrated, based on the frequency of the STR alleles (i.e., variations) in the Caucasian population. The numbers 5-13 represent the STR alleles. The letters A-I represent the SNPs.

It is clear that genetic variants accumulate in an organism's genome over time provided that they do not decrease the fitness of the organism. In the case of STRs the loci used for human identification are specifically chosen for their neutrality within the human genome and therefore variants are by definition neutral with regard to the organism's fitness. If unique SNP patterns can not be found for every STR allele, the SNPs linked to specific groups of alleles can be used. Further, by grouping the SNPs into meta-groups it will be possible to define groups of individuals that are associated together. For example a street gang that has a cultural theme. This will still have strong statistical significance, especially when multiple loci are examined.

In one embodiment, a single SNP pattern can be associated with a single STR allele. In another embodiment, a single STR allele can be associated with more than one SNP pattern. In a further embodiment, a single SNP pattern can be associated with more than one STR allele.

For example, in FIG. 5, the SNP allele B can be associated with STR allele 5, 6, 8, and 9. As another example, an SNP STR-associated genetic pattern can be associated with THO1 6, 7 and 8 but not 9, 10 or 10.1. In one embodiment, doing this type of associating for all 13 CODIS loci, and perhaps others, the CODIS database could be searched for entries that have, for example, ThO—6, 7, 8; VWF—5, 6, 7; D21—11, 12. This could result in selection of a group of individuals who could have contributed the biological sample. Other information (e.g., location of crime, location of individual) could be used to further narrow the number of individuals who might match.

Further, by grouping the SNPs into meta-groups, it can be possible to define groups of individuals that are associated together (e.g., a gang that has a cultural theme, related individuals). Because there is no fitness related constraint on genetic regions used for STR human identification, the SNPs and STRs have filtered through the population from the time that neo-modern human genomes effectively fixed on 23 chromosome pairs. This time frame is long enough to have developed associations as a result of population dispersement. Further, when applied to an evolutionary snapshot of only a few to several generations, one embodiment of the invention is be insulated from additional ongoing mutations. This is because the STR mutation rate, which is greater than the SNP rate (estimated to be 0.01 per generation), is estimated to be only 0.2 per generation. Therefore in 3 generations, it is not likely that an STR allele will mutate. Since forensic applications involve the investigation of living or recently deceased individuals, mutation rate differential between STRs and SNPs will not create an issue. In this way, organisms of several generations can be compared with relative accuracy.

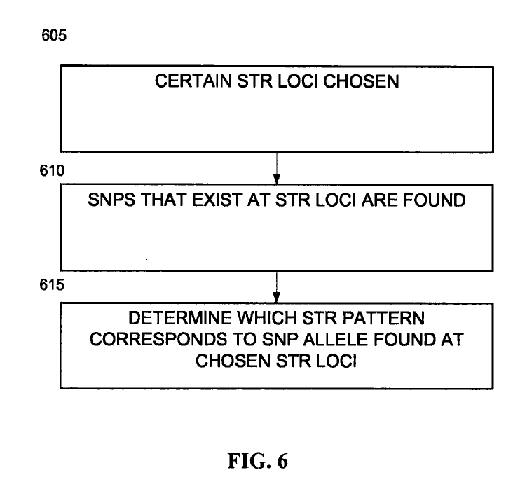

FIG. 6 illustrates details related to how the SNP information can be compared to the STR locus allele information in order to obtain an associative value (see 315 above) indicating the probability that the organism is a match to the biological sample. In 605, a certain STR locus is chosen. In 610, the SNPs that exist at the chosen STR loci are found. In 615, a “Rosetta stone” is used to figure out which STR pattern corresponds to the SNP allele found at the chosen STR loci. FIG. 5 illustrates some STR allele patterns that correspond to the SNP allele, forming the and how an associative value may be applied to infer which STR alleles are likely to associate with a given SNP constellation. FIG. 5 is a highly simplified model of how SNPs may be associated with STRs. For example, from FIG. 4 we see that SNP allele A is associated with STR alleles 5, 6, 7, 8, 9, 10, 11, 12, and 13, By itself, it is not helpful in inferring which STR allele is present in the sample but it does help identify the locus. SNP allele B is associated with STR alleles 5, 6, 8 and 9. Therefore a SNP constellation of A, B would infer the presence of STR alleles 5, 6, 8 and 9 in the sample. Identifying the presence of SNP allele D in the sample would identify the presence of STR allele 9, thereby providing a definite STR allele identification. Note that each loci of interest can have a table similar to FIG. 5, except that each table would likely have several hundred or thousand rows and columns representing the STR and SNP information for each locus of interest.

Returning to FIG. 3, in 320, the associative value can be used to generate at least one SNP genotype database 135. For example, input δ(x) can yield output s(x). δ(x) can represent a function which has the value zero everywhere except at x, where it has the value 1. In addition, s(x) can represent a function, where s is the spread function and x is the molecular weight, measured in base pairs (bp) of the STR system or the array location of the SNP. For each DNA sample input, there can be a set of output electropherograms, represented by nτ(x), where n varies from 1 to n max and can indicate which dye is used for the STR electropherogram (since multiple STR alleles can have the same molecular weight but are different due to the dye) or the array location of the SNP.

In addition, the formula nτ(x)=nΣiaᵢsn(x−xᵢ), where n=1 to k, can be used. nΣi can indicate the summation over the subscript i for the nth dye/SNP electropherogram; sn(x) can denote the spread function of the system for the nth dye electropherogram; xi can be the location of the ith peak in the respective electropherogram (or the SNP array location); ai can be the amplitude of the ith peak; and k can be the number of the SNP system or the dye of the STR system. This formula can be helpful because, in general, the spread functions in each dye electropherogram may be different from the others, and in the case of mixed DNA samples, the amplitudes of the peaks can vary.

In one example, only a single electropherogram is used, and the expansion can include all dyes/SNPs. It is implied that the calculation must be repeated for each dye for single DNA samples aᵢ=aⱼ for all i and j. That is, the amplitudes of all the peaks in a single dye electropherogram are equal. There is an exception when a doublet occurs, but for the case of a single DNS sample, there is no loss of generality in including the secondary peak in the set. The peaks, maxima of the individual spread functions, can be located at the points xᵢ determined by the equation s″(x−xᵢ)=0. Because they are all equal, the amplitudes may be normalized to 1. From nτ(x), the identifier nΩ(x) can be constructed as follows:

nΩ(x)=nΣiδ(x−xᵢ)

Because there are no zero elements in the DNA sample, nΩ(0)=nN, where nN is the number of peaks in the nth dye electropherogram or the array locations of the SNP. Each SNP array therefore can have a unique identifier as will each STR electropherogram. Consequently, there can be one SNP and one STR identifier that exactly correlate with each other and a single individual, and therefore the STR type can be determined by the associative value generated for the SNP. The exact correlation of the two identifiers can be determined empirically.

Returning again to FIG. 3, in 325, the SNP genotype database 135 can be compared with an STR locus allele database 140 to determine if there are any matches. It should be noted that, in one embodiment, the STR locus allele database 140 can contain human STR information; animal STR information (e.g., domestic animals, wild animals, insects, invertebrates); microbe information; or plant STR information; or any combination thereof. In one embodiment, the STR information could be unrelated to forensics (e.g., Genome Wide Association Studies).

Searching a database to find a match to a suspect DNA sample is analogous to searching through a series of messages to determine if a particular signal is embedded in one or more of them, and if so, where it is located. In one embodiment, a search can cross correlate f(x) with each μn(x). If there is a match for f(x) in μn(x), the correlation will peak at its position. Mathematically, this can be represented by:

C(x)=∫f(σ)μ(σ−μ)dσ

In the case of DNA analysis, the signals must have not only the same shape but also the same origin. Furthermore, both f(σ) and μ(σ) are discrete functions. Under these circumstances, the cross correlation integral can be reduced to discrete products and summations.

In 330, if there are any matches, information about the matches can be provided by a match module 145. This can facilitate identification of at least one organism.

Although the invention has been described with reference to the above examples, it will be understood that modifications and variations are encompassed within the spirit and scope of the invention. Accordingly, the invention is limited only by the following claims.

We claim:

1. A method for inferring STR allelic genotype from SNPs in a genome comprising obtaining statistical probabilities for the association of a plurality of SNPs in a genome with at least one Short Tandem Repeat (STR) locus allele for the genome to obtain a SNP constellation association value.

2. The method of claim 1, wherein the SNP constellation association value for a nucleic acid-containing sample is compared with information from a database of STR locus alleles, wherein a match allows identification of an individual from the sample.

3. The method of claim 2, wherein the database contains human STR information.

4. The method of claim 2, wherein the database is selected from the group consisting of STR information from a domestic animal, a wild animal, a plant, an insect, a microbe, and an invertebrate.

5. The method of claim 1, wherein the SNP constellation is used to generate a database of SNP genotypes.

6. The method of claim 1, wherein the at least one STR locus allele comprises one or more CODIS STR loci.

7. The method of claim 2, wherein the sample is a biological sample.

8. The method of claim 7, wherein the sample is selected from the group consisting of blood, semen, vaginal swabs, tissue, hair, saliva, urine, bone, skin and mixtures of body fluids.

9. The method of claim 7, wherein the sample is from a crime scene.

10. The method of claim 7, wherein the sample contains mixtures of human tissue.

11. The method of claim 10, wherein the sample contains tissue from more than one individual.

12. The method of claim 1, wherein the STR loci are selected from the group consisting of CSF1PO, FGA, TH01, TPOX, VWA, D3S1358, D5S818, D7S820, D8S1179, D13S317, D16S539, D18S51, D21S11 D2S1338, D19S433, D1S1656, D2S441, D10S1248, D12S391, D22S1045, SE33, Penta E, and Penta D.

13. The method of claim 6, wherein the CODIS STR loci are selected from the group consisting of TH01, TPOX, CSF1PO, vWA, FGA, D3S1358, D5S818, D7S820, D13S317, D16S539, D8S1179, D18S51, and D21S11.

14. The method of claim 1, wherein the plurality of SNPs are from about 10 to 30,000,000 SNPs.

15. The method of claim 1, wherein the plurality of SNPs are from about 30,000 to 3,000,000 SNPs.

16. The method of claim 1, wherein the plurality of SNPs are from about 300,000 to 3,000,000 SNPs.

17. The method of claim 1, wherein the plurality of SNPs are from about 3,000,000 to 30,000,000 SNPs.

8. The method of 6, further comprising at least one non-CODIS STR locus allele.

19. A method for generating a SNP constellation for a genome comprising obtaining a plurality of SNPs in a genome that are associated with an STR type.

20. A SNP constellation obtained by the method of claim 19.

21. A database containing the SNP constellation of claim 20.

22. A system for inferring STR allelic genotype from SNPs in a genome comprising obtaining statistical probabilities for the association of a plurality of SNPs in a genome with at least one Short Tandem Repeat (STR) locus allele for the genome to obtain a SNP constellation association value and comparing the value with a database of STR locus alleles, wherein the output provides matches allowing identification of an individual from the sample.

23. A method for inferring a genetic variant locus allele in a genome comprising:
obtaining statistical probabilities for the association of a plurality of SNPs in a genome with at least one genetic variant locus allele for the genome to obtain a SNP constellation association value.

24. The method of claim 23, wherein the genetic variant locus allele is an insertion, deletion, repeat variant, copy number variant, translocation, methylation modification, deacetylation modification, or epigenetic marker.

25. The method of claim 24, wherein the genetic variant locus allele is at the locus for amelogenin.

26. The method of claim 24, wherein the genetic variant locus allele is associated with a disease or disorder.

27. A computer system comprising: a relational database having records containing a) information identifying the SNP constellation of claim 20 for a genome; b) information identifying at least one polymorphic locus allele; and c) a user interface allowing a user to selectively access the information contained in the records.

28. The system of claim 27, wherein the polymorphic locus allele is an STR.

29. The system of claim 27, wherein a SNP constellation association value is determined based on a) and b).

30. A computer program product comprising: a computer-usable medium having computer-readable program code embodied thereon relating to a relational database having records containing a) information identifying the SNP constellation of claim 20 for a genome; b) information identifying at least one polymorphic locus allele; wherein a SNP constellation association value is determined based on a) and b).

31. The computer program product of claim 30, comprising computer-readable program code for effecting the following steps within a computing system: providing an interface for receiving a query relating to the information contained in the records; determining matches between the query entry and the information; and displaying the results of the determination.

32. A computerized method for inferring STR allelic genotype from SNPs in a genome comprising:
receiving, by a computer, a plurality of SNPs of the genome;
receiving, by the computer, a STR locus allele of the genome;
computing, by the computer, a SNP constellation association value associating the plurality of SNPs of the genome with the STR locus allele for the genome.

33. The method of claim 32, wherein a database contains the SNP constellation association value.

34. The method of claim 32, wherein the SNP constellation association value is compared with a database of STR locus alleles, and wherein the output provides a match allowing identification of an individual from the sample.

35. The method of claim 34, wherein the following formula is used to generate the output:
nτ(x)=nΣiaisn(x−xi).

36. A computerized method for inferring genetic variant locus allele in a genome, comprising:
receiving, by a computer, a plurality of SNPs of the genome; receiving, by the computer, a SNP constellation association value associating the plurality of SNPS of the genome with the STR locus allele for the genome; and
computing, by the computer, statistical probabilities for the association of a plurality of SNPs in the genome with a genetic variant locus allele for the genome to obtain a SNP constellation association value.

37. The method of claim 36, wherein the genetic variant locus allele is an insertion, deletion, repeat variant, copy number variant, translocation, methylation modification, deacetylation modification; or epigenetic marker; or any combination thereof.

38. The method of claim 37, wherein the genetic variant locus allele is at the locus for amelogenin.

39. The method of claim 37, wherein the genetic variant locus allele is associated with a disease or disorder.

40. A computer system for inferring STR allelic genotype from SNPs in a genome comprising:
a server and a client connected by a network;
an application connected to the server and/or the client by the network, the application configured for:
receiving, by a computer, a plurality of SNPs of the genome;
receiving, by the computer, a STR locus allele of the genome; and
computing, by the computer, a SNP constellation association value associating the plurality of SNPs of the genome with the STR locus allele for the genome.

41. The system of claim 40, further comprising a relational database having records containing: a) information identifying a SNP constellation for a genome; b) information identifying a polymorphic locus allele; and c) a user interface allowing a user to selectively access the information contained in the records.

42. The system of claim 40, wherein the polymorphic locus allele is a STR.

43. The system of claim 40, wherein the SNP constellation association value is determined based on a) and b).

44. A computerized system for inferring a genetic variant locus allele in a genome, comprising:
a server and a client connected by a network;
an application connected to the server and/or the client by the network, the application configured for:
receiving, by a computer, a plurality of SNPs of the genome;
receiving, by the computer, a SNP constellation association value associating the plurality of SNPS of the genome with the STR locus allele for the genome; and
computing, by the computer, statistical probabilities for the association of a plurality of SNPs in the genome with a genetic variant locus allele for the genome to obtain a SNP constellation association value.
